(19) United States
(12) Patent Application Publication
(10) Pub. No.: US 2005/0175669 A1
(43) Pub. Date: Aug. 11, 2005

(54) Highly lubricious hydrophilic coating utilizing dendrimers

(76) Inventors: Oscar Jimenez, Coral Gales, FL (US); Fred Moll, Pembroke Pines, FL (US)

Correspondence Address: WELSH & KATZ, LTD, 120 S RIVERSIDE PLAZA, 22ND FLOOR, CHICAGO, IL, 60606, US

(21) Appl. No.: 11/077,316

(22) Filed: Mar. 10, 2005

Related U.S. Application Data
(62) Division of application No. 10/051,818, filed on Jan. 16, 2002.

(51) Int. Cl. B05D 3/00; A61L 2/00; A61K 31/727

(52) U.S. Cl. 424/423; 427/2.1; 514/56

(57) ABSTRACT

The highly lubricious hydrophilic coating for a medical device comprises a mixture of colloidal aliphatic polyurethane, an aqueous dilution of PVP and specific dendrimers to enhance the physical integrity of the coating, to improve adhesion and to covalently bind or load one of a certain antithrombolitic drug or a certain antibiotic drug or other agent within the dendrimer structure.

### BACKGROUND OF THE INVENTION

1. Field of the Invention

The present invention relates to a highly lubricious hydrophilic coating capable of being applied to the surface of various medical devices such as intravascular catheters, urinary catheters, guidewires, drainage catheters, indwelling catheters, and neuroradiology microcatheters, etc. More specifically, the hydrophilic coating comprises a mixture of colloidal aliphatic polyurethane, an aqueous dilution of PVP and specific dendrimers to enhance the physical integrity of the coating, to improve adhesion and to covalently bind or load certain antithrombolitic drugs such as heparin within the dendrimer structure.

2. Description of the Prior Art

The introduction of medical devices, such as a catheter into the vasculature, is facilitated if the device exhibits a lubricious surface to reduce friction between the percutaneous entry point, vessel wall and catheter materials. In general, catheters are made of a hydrophobic polymeric thermoplastics such as nylon, polyurethane, PVC and other similar plastics. These material substrates do not possess an inherent surface lubricity and, therefore, require the addition of a hydrophilic coating to reduce the coefficient of friction of the catheter.

A lubricious surface helps in crossing coronary lesions in order to facilitate subsequent dilatation of stenotic vessels.

Heretofore, various types of coatings for, and methods of coating, medical devices, such as catheters have been proposed. Examples of analogous and non-analogous coatings and methods are disclosed in the following U.S. Patents.

| PATENT NO. | PATENTEE |
|---|---|
| 3,566,874 | Shepherd |
| 3,598,127 | Wepsic |
| 3,695,921 | Shepherd et al. |
| 4,136,250 | Mueller et al. |
| 5,635,603 | Hansen et al. |
| 5,688,486 | Watson et al. |
| 6,160,084 | Langer et al. |
| 6,242,042 | Goldstein et al. |
| 6,261,271 | Solomon et al. |

### BRIEF DESCRIPTION OF THE SEVERAL VIEWS OF THE DRAWINGS

FIG. 1 is a plan view of a Dendrimer structure;

FIG. 2 is a plan view of a Dendrimer structure loaded with drugs;

FIG. 3 is a plan view of a catheter constructed according to the teachings of the present invention positioned in a blood vessel for heparin elution;

FIG. 4 is a plan view of a Dendrimer reinforced hydrophilic matrix:

FIG. 5 is a plan view of one embodiment of automatic dipping equipment for hydrophilic coating of a medical device such as a catheter.

FIG. 6 is a plan view of a catheter constructed according to the teachings of the present invention and having two hydrophilic coated zones;

FIG. 7 is a top plan view of a hydrophilic coating spray arrangement for coating a catheter.

### BRIEF SUMMARY OF THE INVENTION

As will be described in greater detail hereinafter, the proposed hydrophilic coating is obtained by using a colloidal aliphatic polyurethane resin emulsion and an aqueous dilution of poly (1-vinylpyrrolidone-co-2-dimethylamino ethyl methacrylate) (PVP) in specific ratios to render an acceptable viscosity. The viscosity of this mixture determines the thickness of the applied hydrophilic coating; therefore, titration of the coating mixture viscosity to a specific material substrate will determine the coating thickness, hydrophilicity, adhesion and optimum performance.

The coating is applied to the medical device using a controlled dipping (immersion) process where by the immersion and retraction rates of the device in and out of the coating fluid is controlled using a predetermined displacement rate. Once the dip coating process is completed, the device is allowed to air dry in order to evaporate the remaining fluids. The resulting polymerized (dried) coat is a highly polished, hydrophilic aliphatic polyurethane-PVP film capable of absorbing body fluids to render a highly lubricious surface. Furthermore, the polymerized hydrophilic coating strongly adheres to the substrate even after the body fluids are absorbed. Once hydration of the coating is completed, the coating acquires a translucent appearance that confirms the water absorbtion.

The proposed new hydrophilic coating art utilizes a micromolar concentration of specific dendrimers to provide further cohesive (mechanical) reinforcement and bonding of the hydrophilic matrix.

Another objective of this invention is to bind or load certain pharmacological agents such as sodium heparin within the dendrimer/hydrophilic polymer matrix. Once the hydrophilic coating absorbs the body fluids, the heparin will be eluted from the hydrophilic polymer matrix at predetermined rates for a specific period of time during the medical procedure. This characteristic is important during invasive catheterization procedures such as a percutaneous transluminal coronary angioplasty (PTCA).

### DETAILED DESCRIPTION OF THE INVENTION

Dendrimers are considered a class of artificial molecules discovered by Donald A. Tomalia of the Michigan Molecular Institute in Midland, Mich. Dendrimers (from Greek dendra for tree) are nanoscopic globular molecules about the size of a typical protein; however, dendrimers do not come apart easily as proteins do, because they are held together with stronger chemical bonds. Similar to a cannopy of mature trees, dendrimers contain voids; hence, they have an enormous amount of internal surface area and they can be tailored with smaller or larger internal cavity sizes. Dendrimers are 3-dimensional molecules that are built up from branched units called monomers. A high level of synthetic control is achieved through stepwise reactions, building the dendrimer up one monomer layer, or “generation,” at a time. Each dendrimer starts with a core molecule which is referred to as “generation 0”. Each successive repeat of two sequential reactions forms the next generation, “generation 1,” “generation 2,” and so on until the terminating generation.

Dendrimer's unique architecture has resulted in numerous improved physical and chemical properties when compared to traditional linear polymers as shown in Table A below.

TABLE A
Comparison Between Linear Polymers and Dendrimers.

| Property | Linear Polymer | Dendrimer |
|---|---|---|
| Water Solubility | Low | Very high |
| Shape | Random coil | Spherical |
| Viscosity | High | Low |
| Reactivity | Low | High |
| Surface Polarity | Low | Very High |
| Compatibility | Low | High |
| Compressibility | High | Low |
| Structural Control | Low | Very high |

Dendrimers have two major chemical environments that can be taken advantage of; the high surface functionality/chemistry on the exterior and the voids in the interior of the sphere. The hydrophobic/hydrophilic and polar/nonpolar interactions can be varied in the two environments.

The exterior surface chemistry of the dendrimer may be comprised of several morphologies such as amines, hydroxyl and carboxyl groups among a host of others. The functional groups on the surface are due to either the termination generation or specific chemical modifications to these groups. The sphere's interior, which is largely shielded from exterior environments, comprises voids that have the ability to accept guest molecules; this space functions as the recipient of certain drugs. The existence of two distinct chemical environments in such a molecule makes it possible to use it in applications such as medical device hydrophilic coatings.

Further application, of polyamidoamine (PAMAM, Starburst dendrimers) with either ethylene diamine (E series) or amine (N series) as the core have terminal functional groups comprising, among others, of: —NH₃, —OH, and —COOH or combinations thereof. They provide for novel in vivo controlled release of antithrombogenic and antibiotic drugs as well as applications in enhancing the adhesion of hydrophilic coatings to various substrates via light, pH, and osmotic pressure. This is done by increasing the number of hydrogen bonds, and cationic/anionic interactions between the surface functionality of the dendrimer and that of the aliphatic polyurethane/PVP/water coating fluid.

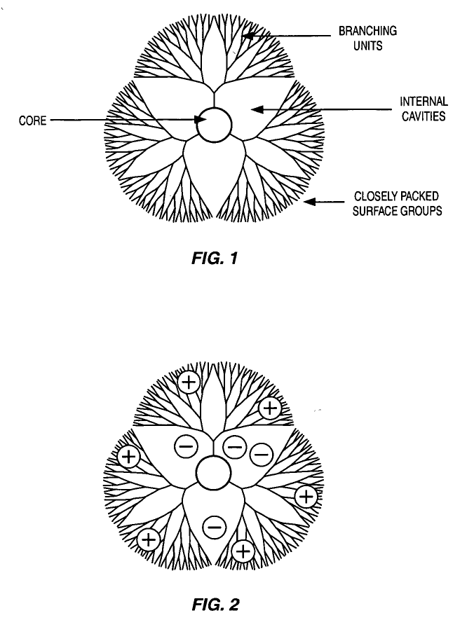

In one example, the voids inside the dendrimer are useful in containing the sodium heparin molecule within the hydrophilic media. The heparin molecule is later eluted from the hydrophilic complex to the body fluids such as blood once the hydrophilic coating is hydrated by body fluids. The elution process continues until the concentration of heparin is near depletion. FIG. 1 shows the voids inside a dendrimer and FIG. 2 shows the drug loaded within the voids.

The elution of antithrombolitic agents such as sodium heparin is important to minimizing blood clotting complications during vascular catheterization procedures. In contrast to systemic injections of heparin, the elution of antithombolitic agents from the surface of the medical device provides the target delivery or release of the drug at the surface of the invasive material. Therefore, a more direct and effective antithrombolitic treatment is administered.

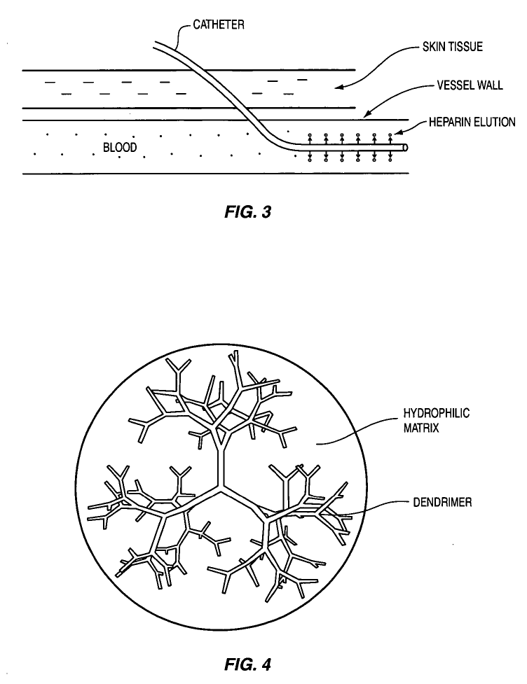

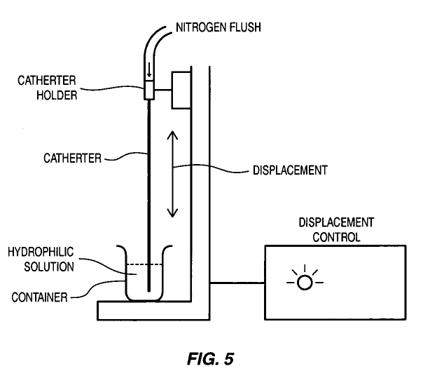

FIG. 3 illustrates the elution of heparin from a catheter after hydration of the hydrophilic coating by body fluids and FIG. 4 illustrates the reinforcement of the hydrophilic coating provided by the dendrimer structure.

The coating is best applied using a dipping process whereby the rate of introduction and retrieval of the medical device is controlled using automatic equipment as illustrated in FIG. 5. The device (catheter) being introduced in the hydrophilic emulsion is flushed with nitrogen to inflate the balloon in order to have a very consistent coating. A guide wire in the catheter is discarded after dipping to prevent the solution from entering the lumen of the catheter.

In another embodiment, the dendrimers in the hydrophilic coating may be loaded with a variety of antibiotic agents. In this configuration, a medical device such as a sheath introducer or indwelling vascular catheter could elute the antibiotic directly to the skin-tissue entry point (proximal segment) in order to prevent infections. The puncture site where the catheter enters the skin is usually vulnerable to bacterial infection.

Each year, as many as 100,000 patients with indwelling vascular catheters become infected, resulting in human suffering and healthcare cost estimated in excess of $300 million (See MDDI, November 2001, page 42).

The incorporation of an antibiotic eluding hydrophilic coating results in a virtually infection resistant device/material that will reduce the incidence of infection.

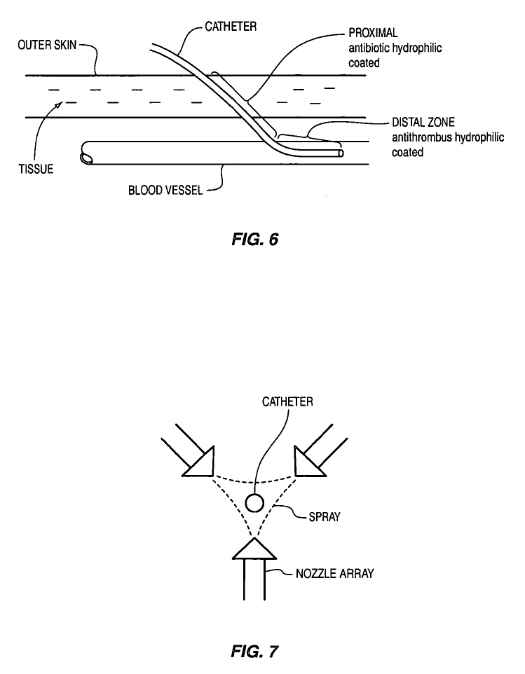

In yet another embodiment, a medical device could be coated with a hydrophilic coat containing an eluting anti-thrombogenic drug in blood contacting areas and an antibiotic drug eluting in other areas where the device comes in contact with tissue, such as the entry point where the medical device penetrates the skin-tissue. This concept is illustrated in FIG. 6.

This dual function hydrophilic coating could be best applied in any medical device that is partially introduced into a blood vessel using a percutaneous approach, that is, where the distal section of the device is inside the body and the the proximal end of the device remains outside the body. The distal segment will exhibit an antithrombolitic drug eluting hydrophilic coating while the proximal segment will exhibit an antibiotic eluting hydrophilic coating.

Another aspect of the invention provides for the integration of both antithrombolitic and antibiotic drugs in the same hydrophilic-dendrimer matrix.

Another method of hydrophilic coating application involves the use of airless spraying on to the medical device. In this method, the medical device is sprayed using an automatic airless spraying system having multiple spray heads as shown in FIG. 7. The medical device is displaced concentric to the spray heads system at a specific rate of speed and later cured by evaporation of the water.

In yet another embodiment, the hydrophilic polymer matrix can be loaded with a biocompatible dye in order to provide a color to the coating. This feature helps in visually inspecting the coating coverage during and after the coating process. Further, an ultraviolet (UV) tracing dye could be added the polymer matrix to render the dye visible only when a UV source is used to illuminate or reveal the coating. The dyes are loaded to the dendrimers in a similar manner as shown in FIG. 2.

The hydrophilic coating formulation is obtained by colloidal dispersion of an aliphatic polyurethane polymer in a solvent mixture as follows:
Aliphatic polyurethane polymer
Purified Water
N-methyl-2 Pyrrolidone
Dendrimer
Poly (1-vinylpyrrolidone-co-2-diamethylamino ethyl methacrylate)-PVP Triethylamine
Sodium heparin and/or antibiotic drugs and/or dye

The coating components are mixed and dispersed in specific proportions to render a suitable viscosity fluid. The final coating formulation yields an aqueous colloidal dispersion of a polymer intended for medical device hydrophilic coating. Such gelatinous hydrophilic coatings on various medical devices permits release of pharmacological agents.

From the foregoing description, it will be apparent that the method and device of the present invention have a number of advantages, some of which have been described above and others of which are inherent in the invention.

Also, it will be understood that modifications can be made to the method and device of the present invention without departing from the teachings of the invention. Accordingly, the invention is only to be limited as necessitated by the accompanying claims.

1. A catheter having an outer surface and said outer surface having an highly lubricious hydrophilic coating comprising: a mixture of colloidal aliphatic polyurethane, an aqueous dilution of PVP and dendrimers selected from the group consisting of: poly(amidoamine dendrimers, poly(propylene imine) dendrimers, polyether dendrimers, Phenlyacetylene dendrimers, chiral dendrimers, and tecto dendrimers, to enhance the physical integrity of the coating, to improve adhesion and to covalently bind or load an antibiotic drug or anti-thrombolytic drug selected from: tissue plasminogen activator; alteplase, recombinant; anistreplase; reteplase, recombinant; streptokinase; or urokinase within the dendrimer structure.

2-4. (canceled)

5. The catheter of claim 1 wherein said coating comprises a colloidal dispersion of an aliphatic polyurethane polymer in a solvent mixture including:
an aliphatic polyurethane polymer;
purified water;
N-methyl-2 pyrrolidone;
dendrimers;
poly (1-vinylpyrrolidone-co-2-diamethylamino ethyl methacrylate)-PVP triethylamine; and,
said antibiotic drug or anti-thrombolytic drug

6-9. (canceled)

10. A method for applying the coating of claim 1 to the catheter comprising the step of dipping the catheter into a solution containing the mixture of colloidal aliphatic polyurethane, the aqueous dilution of PVP and the dendrimers.

11. A method for applying the coating of claim 1 to the catheter comprising the step of airless spraying of the catheter with a solution containing the mixture of colloidal aliphatic polyurethane, the aqueous dilution of PVP and the dendrimers.

12. A method for applying the coating of claim 1 to the catheter comprising the step of dipping the catheter into a solution containing the mixture of colloidal aliphatic polyurethane, the aqueous dilution of PVP and the dendrimers.

13. The method of claim 12 further including the step of flushing a lumen of the catheter with nitrogen during the dipping process to prevent the solution from entering the catheter's lumen.

14. A catheter coated, in a first zone where the catheter contacts blood, with a first hydrophilic coating containing an eluting anti-thrombolytic drug, and coated, in a second zone, where the catheter comes in contact with tissue, with a second hydrophilic coating containing an eluting antibiotic drug.

15. The catheter of claim 14 wherein each hydrophilic coating comprises a mixture of colloidal aliphatic polyurethane, an aqueous dilution of PVP and dendrimers to enhance the physical integrity of the coating, to improve adhesion and to covalently bind or load with one of the anti-thrombolytic drug or the antibiotic drug.

16. (canceled)

17. A guidewire having an outer surface and said outer surface having an highly lubricious hydrophilic coating comprising: a mixture of colloidal aliphatic polyurethane, an aqueous dilution of PVP and dendrimers selected from the group consisting of: poly(amidoamine dendrimers, poly(propylene imine) dendrimers, polyether_dendrimers, phenlyacetylene dendrimers, chiral dendrimers, and tecto dendrimers, to enhance the physical integrity of the coating, to improve adhesion and to covalently bind or load an antibiotic drug or anti-thrombolytic drug selected from: tissue plasminogen activator: alteplase, recombinant; anistreplase; reteplase, recombinant; streptokinase; or urokinase within the dendrimer structure.

18. The guidewire of claim 17 wherein said coating comprises a colloidal dispersion of an aliphatic polyurethane polymer in a solvent mixture including:
an aliphatic polyurethane polymer;
purified water;
N-methyl-2 pyrrolidone;
dendrimers;
poly (1-vinyl pyrrolidone-co-2-diamethylamino ethyl methacrylate)-PVP triethylamine; and,
said antibiotic drug or anti-thrombolytic drug.

19. A method for applying the coating of claim 17 to a guidewire comprising the step of dipping the guidewire into a solution containing the mixture of colloidal aliphatic polyurethane, the aqueous dilution of PVP and the dendrimers.

20. A method for applying the coating of claim 17 to the guidewire comprising the step of airless spraying of the guidewire with a solution containing the mixture of colloidal aliphatic polyurethane, the aqueous dilution of PVP and the dendrimers.

21. A method for applying the coating of claim 18 to the guidewire comprising the step of dipping the guidewire into a solution containing the mixture of colloidal aliphatic polyurethane, the aqueous dilution of PVP and the dendrimers.

23. A guidwire coated, in a first zone where the guidewire contacts blood, with a first hydrophilic coating containing one of an eluting anti-thrombolytic drug, and coated, in a second zone, where the guidewire comes in contact with tissue, with a second hydrophilic coating containing an eluting antibiotic drug.

24. The guidewire of claim 23 wherein each hydrophilic coating comprises a mixture of colloidal aliphatic polyurethane, an aqueous dilution of PVP and dendrimers to enhance the physical integrity of the coating, to improve adhesion and to covalently bind or load with one of the anti-thrombolytic drug or the antibiotic drug.
